(19) United States
(12) Patent Application Publication
(10) Pub. No.: US 2005/0265926 A1
(43) Pub. Date: Dec. 1, 2005

(54) Methods and reagents for improving localization of treatment and visualization ligands to sites in a mammal

(76) Inventor: Meir Strahilevitz, Seatle, WA (US)

Correspondence Address: POLSTER, LIEDER, WOODRUFF & LUCCHESI, 12412 POWERSCOURT DRIVE SUITE 200, ST. LOUIS, MO, 63131-3615, US

(21) Appl. No.: 11/137,591

(22) Filed: May 24, 2005

Related U.S. Application Data
(60) Provisional application No. 60/574,834, filed on May 27, 2004.

(51) Int. Cl. A61K 49/00; A61K 9/127

(52) U.S. Cl. 424/9.6; 424/450

(57) ABSTRACT

A treatment method comprising administration of visualization ligands or treatment ligands in a ligand-encapsulating vehicle, such as a liposome, preceded by administration of a “cold” vehicle, such as a liposome, lacking visualization ligands and treatment ligands. The cold vehicle accumulates in the reticulo-endothelial system of the mammal to which it is administered and minimizes the accumulation of the ligand-encapsulating vehicle carrying the visualization ligands or treatment ligands in the reticulo-endothelial system, thus reducing the toxicity of the ligands on the reticulo-endothelial system.

### CROSS-REFERENCE TO RELATED APPLICATIONS

This application claims the benefit of provisional application Ser. No. 60/574,834, filed May 27, 2004, the disclosure of which is hereby incorporated by reference herein.

### STATEMENT REGARDING FEDERALLY SPONSORED RESEARCH

Not Applicable.

### BACKGROUND OF THE INVENTION

The present invention relates to targeting of ligands by the administration of liposome-incorporated treatment ligand (TL) or visualization ligand (VL).

The administration may be with or without an additional step of removal, including extracorporeal removal, of the targeted ligands, following the administration of liposome containing VL or liposome containing TL.

To increase the effectiveness of delivering TL or VL via liposome entrapment, the liposomes themselves may be targeted by means of species attached to the walls of the liposomes. Targeted liposomes may include, for example, any of the targeting molecular species disclosed in incorporated applications WO 96/37516 and WO 03/090781 A1, including but not limited to: antibodies; antibody fragments, including synthetic and those produced by genetic engineering; hormones; enzymes; drugs with affinity to target sites in the organism as well as cytokines and any small molecule (below MW 10,000 D) and large molecule (MW above 10,000 D) that are known to have affinity to a target site in the organism.

Targeting of VL or TL by incorporating them in liposomes (or other ligand-encapsulating vehicle) has been hampered by the premature breakdown of the liposome (vehicle) and by clearance of the liposome (vehicle) from the bloodstream before delivery of the ligand to the desired site in a mammal.

H. P. Weckenmann et al. (Arzneim-Forsch./Drug Res. 39 (I), Nr. 3, pp 415-420, 1989, incorporated by reference herein) reported that when the liposomes are targeted liposomes, such as immunoliposomes, after administration of immunoliposomes intravenously to mice, more then 40% of the administered liposomes are eliminated from the blood very rapidly (within minutes). It was proposed that this rapid elimination is caused predominantly by the accumulation of targeted liposome in the reticulo-endothelial system (RES) (primarily liver and spleen). The authors observed that larger immunoliposomes are cleared more rapidly, and clearance is associated with increase in liposome size.

### SUMMARY OF THE INVENTION

In accordance with the present invention, administration of TL or VL contained in liposomes or other ligand-encapsulating vehicles can further be optimized and improved by the addition of an additional step, prior to the administration of the liposome-incorporated ligand. The optimization step can be utilized in conjunction with the use of ligand-containing non-targeted liposomes as well as ligand-containing targeted liposomes.

The optimization of targeting of VL or TL to target sites, in particular cancer target sites, can be utilized, both when the liposome-incorporated VL or TL are bound to a targeting species, and when the liposome-incorporated VL or TL are not bound to a targeting species. Targeting species, in accordance with the current invention include antibodies, antibody fragments, hormones, drugs, enzymes, cytokines or any other targeting species, including, but not limited to the targeting species detailed in the current application.

Thus, the improvement of targeting can be achieved by the use of an optimization step that is performed prior to the administration of liposomes containing a VL or TL.

The optimization step (hereafter referred to as SI) is particularly useful whenever the liposomes containing the VL or TL are themselves targeted (use of targeted liposomes, for ligand targeting) whether or not the ligand itself is also targeted by binding to it, covalently or non-covalently, a targeting species.

The improvement of the targeting, in accordance with the present invention, is achieved by incorporating in the treatment scheme an initial step of administering liposomes that do not incorporate VL or TL, hereafter referred to as “Cold Liposomes” (CL). More generally, the invention encompasses prior administration of any ligand-encapsulating vehicle lacking significant quantities of visualization ligands or treatment ligands in order to minimize the accumulation of the ligand-encapsulating vehicle carrying the visualization ligands or treatment ligands in the reticulo-endothelial system, thus reducing the toxicity of the ligands on the reticulo-endothelial system.

Preferably CL comprise immunoglobulins or other molecules which are bound, preferably covalently, to the liposome wall. These molecules increase the uptake of the CL by macrophage cells of the RES and by kupfer cells in the liver.

CL are preferably selected to comprise liposomes that are known to be effectively cleared by uptake by macrophages of the RES. The immunoglobulins which are bound to the liposomes can be non-specific immunoglobulins such as IgG or IgM or another immunoglobulin class, or the liposome-bound species can be another macromolecule, such as a protein, peptide, polysaccharides, or other polymers. The liposome-bound species can also be a small molecule, with molecular weight below 2000, when such molecules, when bound to the wall of the liposome, increase the uptake of the CL by the RES. When the molecule is an immunoglobulin it does not have to be a specific antibody, but specific antibodies with defined antibody specificity can also be used, if desired. For example, the specific antibodies bound to the wall of the CL, when the treated mammal is a rat, can be a monoclonal antibody (mab) specific to rat macrophages. Such antibodies are available commercially from Cell Sciences, Canton, Mass. When the treated mammal is a species other then a rat, antibodies specific to the macrophages and/or Kupfer cells of the treated species, preferably mabs, are used. When the treated mammal is a human, preferably humanized mabs are used, most preferably human mabs (Ghose, infra, p. 17); these can be produced by those skilled in the art. Alternatively, the molecules bound to the liposome wall can be small molecules or macromolecules that are ligands specific to a receptor on macrophages and/or Kupfer cells of the treated species.

The CL are administered to the treated mammal preferably 5 minutes to 10 hours prior to the administration of the liposome-incorporated VL or TL. Most preferably, they are administered 10 minutes to 3 hours prior to the administration of the liposome-incorporated VL or TL, in the treatment step (SII), in accordance with examples 1-3 of the current application. The number of CL administered in the first step will preferably be 20% to 500% of the amount of targeted liposomes administered in the following targeted treatment step. Most preferably, the amount is 50% to 200%.

The CL used in SI, in accordance with the current invention, preferably have a composition that is associated with increased clearance by the RES. Such liposomes are known in the art and their physical and chemical characteristics are detailed for example in Weckenmann et al., supra, as well as in H. P. Weckenmann, et al., Arzneim-Forsch/Drug Res., 38/II, Nr 11, pp 1556-63, 1988, and in Paul Tardi, et al., in “Cancer Drug Discovery and Development: Tumor Targeting in Cancer Therapy” edited by M Pagé, pp 119-135, Humana Press, Inc., Totowa, N.J., 2002. All of the above publications are incorporated herein by reference.

The following additional sections of the above book edited by M. Pagé relevant to the subject matter hereof are also incorporated herein by reference: T. Ghose, pp. 3-78, in particular pp. 37-38, M. Singh, pp 151-164, in particular, pp 157-160 and p 163, C. M. Allen, et al., pp 329-361, in particular, pp. 337-339, A. S. Lübbe et al., pp 379-388, in particular, pp. 382-384. The following publications are also incorporated by reference herein: M. K. Robinson, et al., Drug Development Research, Vol. 61, pp 172-187, 2004, and Kathleen F. Pirollo, et al., “Immunoliposomes: A targeted delivery tool for cancer treatment” in: “Vector Targeting of Therapeutic Gene Delivery,” edited by David T. Curiel and Joanne T. Douglas, Wiley-Liss, Inc., pp. 33-62, 2002.

The characteristics of liposomes that are cleared rapidly by the RES and are detailed in Tardi, et al., include large size (above 500 nm in diameter) liposomes with charged surface, particularly negatively charged liposomes, for example liposomes containing negatively charged lipids, such as lipids containing phosphatidyl serine, phosphatic acid or cardiolipin are preferred for use as CL, in the first step of treatment aimed at saturation (blocking) of the RES, in accordance with the current invention.

Liposomes that are cleared slowly by the RES are preferred for and are suitable for targeting incorporated VL or TL in the Treatment Step (SII), in accordance with the current invention. Such liposomes include liposomes that contain gangliosides and liposomes that contain polymers, such as Polyethylene Glycol (PEG), amphiphilic polyacrylamide, oligosaccharides and polysaccharides.

Specifically suitable liposomes that can be used as CL, include for example liposomes with composition cholesterol 45 mol %, dipalmitoyl-phosphatydyl-ethanolamine N-hydroxypyridyidithiopropionate (DPPE-PDP) 45 mol %, produced in accordance with Weckenmann et al., supra, Arzneim-Forsch/Drug Res., 38/II, Nr 11, pp 1556-63, 1988. Suitable for use as CL include, for example, batches 37-40 and 52-53 as produced and described in Weckmann et al., supra. When CL are produced they are coupled to antibody IgG or non-antibody IgG of a different mammalian species than the mammal treated, or alternatively, coupled to non-antibody IgG of the same mammalian species that is being treated. For example, if the treated mammal is a human, the CL coupled IgG is human IgG. Optionally, the coupled IgG may be antibody IgG wherein the antibody is preferably humanized antibody (complementary-determining regions CDRs grafted onto a human antibody), and most preferably the mabs are human mabs (Ghose, supra, p. 17), whenever such human mabs are available or can be produced. The antibodies are preferably specific to antigenic determinants on macrophages or Kupfer cells. The immunoglobulin coupled to the CL, whether non-antibody immunoglobulin or antibody immunoglobulin, may be of other immunoglobulin classes such as IgM, IgD, or IgA. The macromolecules coupled to the CL (in order to increase its clearance in the RES), may alternatively be macromolecules other than proteins, including but not limited to peptides, polysaccharides, and other macromolecules. Typically the CL liposomes should contain an average of between 2-30 IgG molecules per liposome and preferably 5-30 molecules per liposome.

The second step (SII) of the treatment of administration (such as by intravenous, intraperitoneal or other route of administration) of targeted liposome incorporated VL or TL is started after completion of SI. The targeted liposomes are preferably administered to the mammal in SII 5 minutes to 10 hours following the completion of SI. Most preferably SII starts 10 minutes to 3 hours following the completion of SI.

An additional step of removal, including extracorporeal removal, of the targeted ligands, following the administration of liposome containing VL or liposome containing TL may be carried out in accordance with known techniques. The optional removal step may be of free targeted ligands, released from liposomes upon the disintegration of the liposomes, or, when desired, removal may include, or be limited to, the removal of liposomes that were not disintegrated and that incorporate the targeted ligands.

To increase the effectiveness of delivering TL or VL via liposome entrapment, the liposomes themselves may be targeted by means of species attached to the walls of the liposomes. Targeted liposomes may include, for example, any of the targeting molecular species disclosed in incorporated applications WO 96/37516 and WO 03/090781 A1, including but not limited to: antibodies; antibody fragments, including synthetic and those produced by genetic engineering; hormones; enzymes; drugs with affinity to target sites in the organism as well as cytokines and any small molecule (below MW 10,000 D) and large molecule (MW above 10,000 D) that are known to have affinity to a target site in the organism.

The reagents required for carrying out SI and SII are preferably supplied as combinations or kits containing the CL (or other ligand-encapsulating vehicle lacking significant quantities of VL or TL) and the liposomes (or other ligand-encapsulating vehicle) containing VL or TL.

### DESCRIPTION OF THE PREFERRED EMBODIMENTS

In one example of the utilization of the current invention:

Following the completion of SI as described above, SII is done in accordance with examples 1 of WO 96/37516 with the following modifications: The targeting antibody specific to carcino embryonic antigen (CEA) MAb ZCE-025 or its Fab fragment is bound to the wall of the targeted liposome and nitrobenzyl-EDTA labeled with ¹¹¹Indium is incorporated in the above targeted liposomes. The targeted liposomes administered to the mammal, when the mammal is a human contain 10 mCi of ¹¹¹Indium. The administration of the targeted liposomes is done typically intravenously, but can be done by other routes when indicated.

In Example 1 above as well as in any other of the examples of the utilization of the current invention:

(A) The step of extracorporeal adsorption (or removal of VL or TL by affinity binding or other removal means that do not include extracorporeal affinity removal) is optional and can be omitted.

(B) The liposomes in which the VL or TL are incorporated that are utilized in SII, optionally can be non-targeted liposomes (they may have no targeting antibody or other targeting molecule bound to their surface).

(C) The CL used in SI, may optionally be liposomes with diameter of 170 nm or larger, that do not have any immunoglobulin or any other protein, peptide or non-protein macromolecule attached to their surface.

(D) Targeted liposomes may include any of the targeting molecular species disclosed in incorporated applications WO 96/37516 and WO 03/090781 A1, including but not limited to: antibodies; antibody fragments, including synthetic and those produced by genetic engineering; hormones; enzymes; drugs with affinity to target sites in the organism as well as cytokines and any small molecule (below MW 10,000 D) and large molecule (MW above 10,000 D) that are known to have affinity to a target site in the organism, wherein the target site is selected for targeting a VL or TL in accordance with the current invention.

(E) In SI and SII, ligand-encapsulating vehicles, known in the art, can replace liposomes, in all of the examples of the current application. Such vehicles include microcapsules and nanospheres (S. Bennis et al., Eur. J. Cancer, 1994: 30 A, pp. 89-93).

Nanoparticles, in particular unmodified nanoparticles can be used in SI and nanoparticles, in particular modified nanoparticles can be used in SII. Modifications of liposomes and other nanoparticles, that lead to reduced clearance (reduced uptake by the RES) are detailed in C. M. Allen et al., supra.

With consideration of A-E above, SI of the current invention can be incorporated into any of the following EXAMPLES:

Examples 1-3 of WO 96/37516.

Examples 1-19, 53-56, 67-70, 72-75 and 82 of WO 03/090781 A1.

The reagents required for carrying out SI and SII are preferably supplied as combinations or kits containing the CL and the liposomes containing VL or TL.

All of the literature references, patents, and patent applications mentioned above are hereby incorporated by reference.

As various changes could be made in the above constructions without departing from the scope of the invention, it is intended that all matter contained in the above description or shown in the accompanying drawings shall be interpreted as illustrative and not in a limiting sense.

1. In combination a first pharmaceutically acceptable ligand-encapsulating vehicle preparation comprising a species selected from a treatment ligand and a visualization ligand incorporated in the ligand-encapsulating vehicle and a second pharmaceutically acceptable ligand-encapsulating vehicle preparation, wherein the second ligand-encapsulating vehicle preparation does not comprise a treatment ligand or a visualization ligand.

2. A method of delivering a ligand to a site in an organism, comprising a first step of administering to the organism the second ligand-encapsulating vehicle preparation of claim 1 followed by a second step of administering to the organism the first ligand-encapsulating vehicle preparation of claim 1.

3. A method of delivering a ligand to a site in an organism, comprising a first step of administering to the organism a first pharmaceutically acceptable ligand-encapsulating vehicle preparation substantially free of treatment ligands and visualization ligands, followed by a second step of administering to the organism a second pharmaceutically acceptable ligand-encapsulating vehicle preparation comprising a species selected from a treatment ligand and a visualization ligand incorporated in the ligand-encapsulating vehicle.

4. The method of claim 3 wherein the site in the organism is a cancer site.

5. The method of claim 3 wherein the site in the organism is a specific receptor in the organism.

6. The method of claim 3 wherein the site in the organism is an infectious agent selected from viruses, bacteria and protozoal agents.

7. The method of claim 6 wherein the infectious agent is an HIV virus.

8. The method of claim 3 wherein the first ligand-encapsulating vehicle preparation consists essentially of empty liposomes.

9. The method of claim 3 wherein the first ligand-encapsulating vehicle preparation comprises liposomes having macromolecules bound to their walls.

10. The method of claim 3 wherein the second step is performed 10 minutes to 3 hours after the first step.

11. The method of claim 3 wherein the first and second ligand-encapsulating vehicle comprise liposomes, the number of first liposomes being 20% to 500% of the number of second liposomes.

12. A kit comprising a first pharmaceutically acceptable ligand-encapsulating vehicle preparation comprising a species selected from a treatment ligand and a visualization ligand incorporated in the ligand-encapsulating vehicle and a second pharmaceutically acceptable ligand-encapsulating vehicle preparation substantially free of treatment ligands and visualization ligands.

13. The kit of claim 12 wherein at least one of the first ligand-encapsulating vehicle and the second ligand-encapsulating vehicle comprises liposomes.

14. The kit of claim 12 wherein the second ligand-encapsulating vehicle comprises liposomes.

15. The kit of claim 14 wherein the liposomes comprise molecules bound to the walls of the liposomes.

16. The kit of claim 15 wherein the molecules are macromolecules.

17. The kit of claim 15 wherein the molecules are ligands specific to at least one of macrophages and Kupfer cells.

18. The kit of claim 17, wherein the molecules are macromolecules.

19. The kit of claim 15 wherein the liposomes comprise antibodies specific to antigenic determinants on macrophages or Kupfer cells.

20. The kit of claim 14 wherein the liposomes have a mean diameter of at least 170 nm.

21. The method of claim 2, further comprising a third step, following the second step, the third step comprising removal of at least one of the first ligand-encapsulating vehicle and the species released from the first ligand-encapsulating vehicle.

22. The method of claim 21 wherein the third step comprises adsorption in an extracorporeal device.

23. The method of claim 3, further comprising a third step, following the second step, the third step comprising removal of at least one of the second ligand-encapsulating vehicle and the species released from the second ligand-encapsulating vehicle.

24. The method of claim 23 wherein the third step comprises adsorption in an extracorporeal device.
